(19) United States
(12) Patent Application Publication
(10) Pub. No.: US 2011/0286882 A1
(43) Pub. Date: Nov. 24, 2011

(54) Automated infection control surfaces

(75) Inventor: Allan Yang Wu, (US)

(73) Assignee: Allan Yang Wu

(21) Appl. No.: 12/800,693

(22) Filed: May 20, 2010

(51) Int. Cl. A61L 2/08; A61L 2/24

(52) U.S. Cl. 422/22; 422/114

(57) ABSTRACT

The invention is an automated infection control surface, which may be placed on the sole of a shoe or on the ground in the form of a floor mat/tile. The invention is composed of a series of individual light sources or LEDs (light emitting diodes) capable of emitting ultraviolet sanitizing light. When in the format of a shoe, there is an electronic switch in the form of a pressure sensor which is triggered when the operator's foot is planted within the shoe and standing firmly on the ground. When the pressure sensor is triggered the electrical current passes from the power source to the light sources or LEDs and illumination of sanitizing light occurs. When the shoe is lifted off the ground and when there is no weight or pressure within the shoe, the pressure sensor is turned off and the LEDs turn off. In this way the shoe is only capable of triggering UV microbicidal activity when the sole is flush to the ground, thus preventing inadvertent UV light scatter and potential skin and eye damage. The invention may also be composed in an array like fashion in the form of a floor mat or tile, such that the LEDs are oriented upwardly to shine towards the sole of the operator's shoe, but always concealed underneath the shoe. In either embodiment the LEDs are turned off when there is no weight on the sole of the shoe or floor mat/tile. Both embodiments of the invention could also be used simultaneously to provide maximum sterilization activity.

### BACKGROUND OF THE INVENTION

PATENTS CITED:

| Patent Number | Title | Issue date |
|---|---|---|
| 5,500,635 | Products incorporating | Mar. 19, 1996 |
| | piezoelectric material | |
| 6,280,045 | Lighted footwear module | Aug. 28, 2001 |
| | with random time delay | |
| 5,978,996 | Shoe tree assembly | Nov. 9, 1999 |

Shoes and flooring sanitation present some of the greatest challenges to infection control. Cleaning floors with a mop along with peroxide, bleach or various detergents is a common means of minimizing the spread of contamination. Shoe covers are also another means of preventing contamination spread. Though both methods are successful current standards of sanitation, both require strict compliance, which can be difficult to obtain in practice, especially in a busy occupational environment.

Another potential solution to this problem is use of ultraviolet light. Antimicrobial lamps utilizing wavelengths in the ultraviolet (UV) spectrum are capable of inducing DNA single strand breaks. For this reason UV light has been used as a cost-efficient and passive means for microbicidal and infection control by flooding whole rooms and surfaces with the active light. However, the use of UV microbicidal lamps is limited, due to the fact that the human eye and skin is susceptible to cataract and skin cancer formation. Current standard of use involves UV lamp irradiation in the absence of human exposure. Thus, UV lamps can only be employed when a room is vacant of personnel. Certainly an invention capable of addressing infection control related to the floor and shoes utilizing UV light, but at the same time avoiding the damaging aspects to the skin or eyes would be of great utility.

### SUMMARY OF THE INVENTION

The invention is an infection control surface on the sole of a shoe, whereby the shoe is composed of a series of UV LEDs (light emitting diodes) on the sole of the shoe. Within the shoe itself is a pressure sensor that is triggered when the foot is planted within the shoe and standing firmly on the ground. When the pressure sensor is triggered the LEDs are turned on. When the shoe is lifted off the ground and when there is no weight or pressure within the shoe, the sensor is turned off and the LEDs turn off. In this way the shoe is only capable of triggering UV microbicidal activity when the sole is flush to the ground, thus preventing inadvertent UV light scatter and potential skin and eye damage.

### DISTINCTION FROM PRIOR ART

The current invention is different from other LED or light accessorized shoes (U.S. Pat. Nos. 5,500,635, 6,280,045) in that the LEDs/light source of the current invention are intentionally hidden visually when in operation. In other words, prior art (U.S. Pat. Nos. 5,500,635, 6,280,045) are intended for direct visualization and aesthetics or illumination, not light concealment and nor were they considered for the utilization of infection control or sterilization use. Other prior art, such as U.S. Pat. No. 5,978,996 utilize an ultraviolet light source for antimicrobial properties, but only in the form of a shoe tree and only for the sterilization of the internal surface of the shoe when the operator is not actively using the shoe.

The invention may also be composed in the form of a floor mat or tile, such that the LEDs/light source are oriented in an upward fashion to shine towards the sole of the operator's shoe, but always concealed underneath the shoe to prevent exposure to the operator.

### BRIEF DESCRIPTION OF THE DRAWINGS

FIG. 1: Conceptual drawing of prior art.

FIG. 2: Conceptual drawing of invention, longitudinal cross-section.

FIG. 3: Conceptual drawing of invention, in a bottom-up view of the shoe sole.

FIG. 4: Conceptual drawing of alternate embodiment of invention in the form of a floor mat.

FIG. 5: Conceptual drawing of single unit of alternate embodiment unactivated.

FIG. 6: Conceptual drawing of single unit of alternate embodiment activated.

FIG. 7: Conceptual drawing of invention in standard and alternate embodiment being used simultaneously.

### DETAILED DESCRIPTION

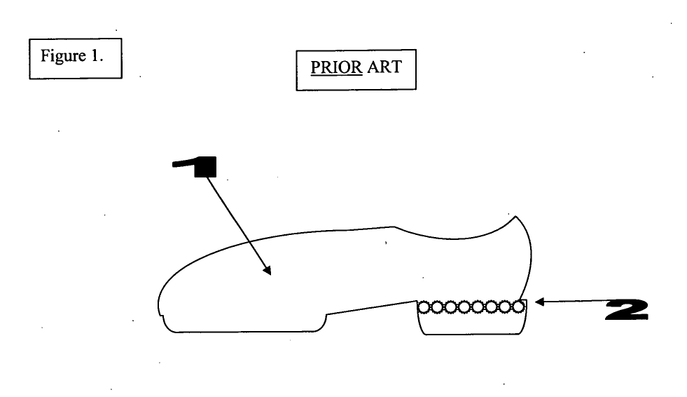

FIG. 1: Conceptual drawing of prior art. Such that a shoe (1) is illuminated by a light source or LEDs (2) in such a format that is visibly conspicuous to the naked eye.

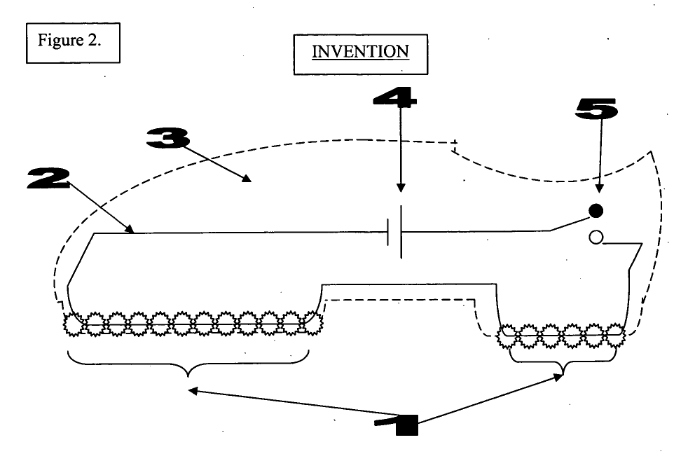

FIG. 2: Conceptual drawing of invention, longitudinal cross-section. Such that the shoe (3) is composed of an internal circuit (2) connected to a power source (4), a weight sensitive switch (5), which when deployed illuminates lights/LEDs (1) connected to the circuit (2).

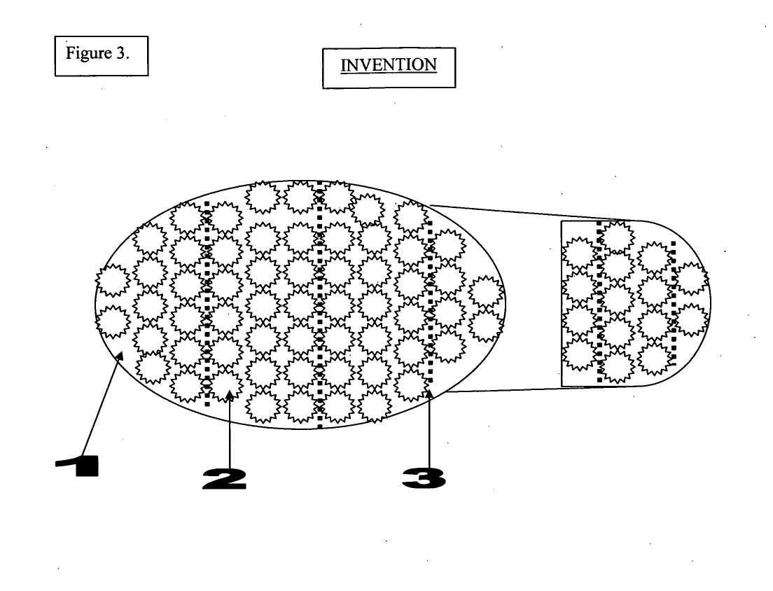

FIG. 3: Conceptual drawing of invention, in a bottom-up view; whereas the sole (1) of the shoe is covered with the light source/LEDs (2) and interspersed grips (3), represented by a series of interspersed parallel dotted lines, are used for traction.

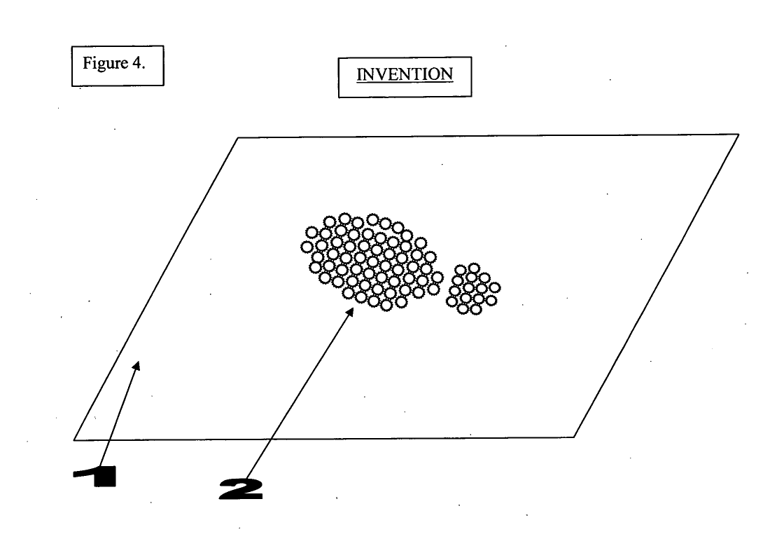

FIG. 4: Conceptual drawing of alternate embodiment of invention, whereas the self-sterilizing touch sensitive surface is placed on a flooring tile or floor mat (1) and the light source or LEDs (2) are aimed upward toward the ceiling, but because invention illuminates in regions where touch/pressure/weight is sensed, the sterilizing light will be concealed beneath the feet of the operator as represented by the footprint silhouette (2).

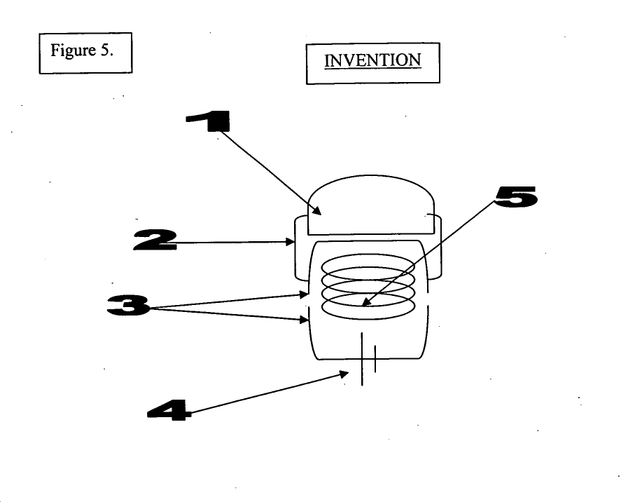

FIG. 5: Conceptual drawing of single unit of alternate embodiment unactivated, such that the LED/light source (1) is electronically connected (2) to an open circuit (3) which is held apart by an insulated or non-conducting spring (5). The open circuit (3) is also connected to a power source such as a battery as in (4).

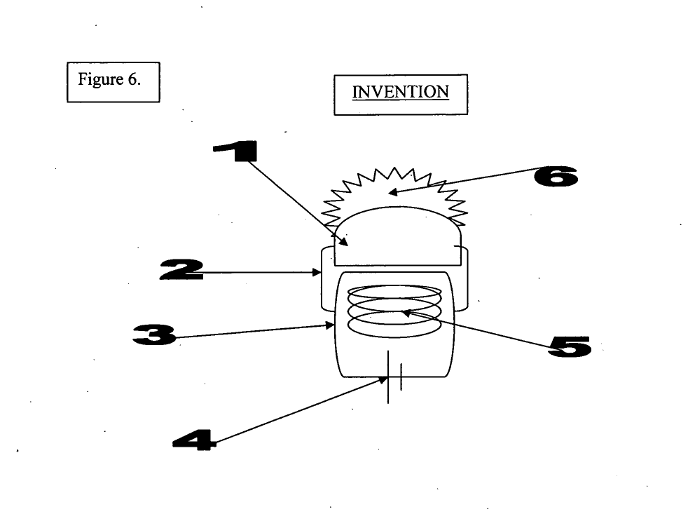

FIG. 6: Conceptual drawing of single unit of alternate embodiment activated by weight being place atop the LED/light source (1), such that the insulated or non-conducting spring (5) is compressed and the circuit is now closed (3) allowing the flow of electrical current from the battery (4) to the LED/light source (1) which is exhibited by shining sanitation light (6).

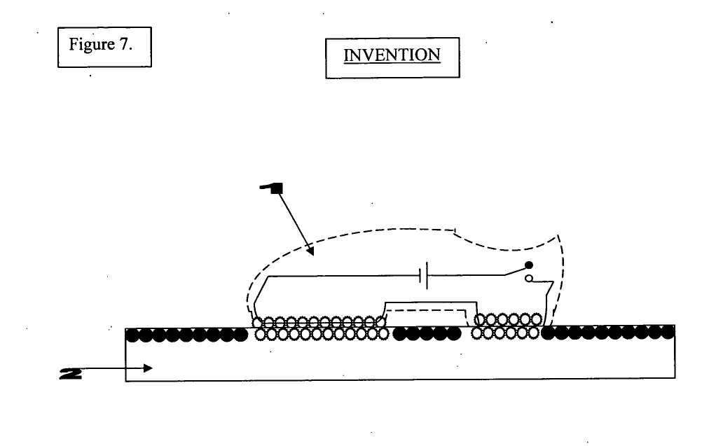

FIG. 7: Conceptual drawing of invention in standard and alternate embodiment being used simultaneously, such that the invention in the form of a shoe (1) stands upon the invention in the form of a floor mat (2). In this way two dual sterilizing surfaces will work in combination to provide even further sterilization of surfaces, but only in the distribution of where the operator is standing and applying weight to trigger the switch and light sensors as indicated by the discrepancy in light sources/LEDs in the off position (black circles) versus light sources/LEDs that have been activated (open star bursts).

I claim:

1. A method in which:
a. an operator wearing the invention in the form of a specialized shoe can simultaneously sterilize the opposing surfaces of the floor and sole of the shoe which are in direct contact with one another, such that:
i. the weight of the operator triggers a switch, within the sole of the shoe that completes an electronic circuit to provide current to Light Emitting Diodes (LEDs) or a light source embedded within the sole which shines directly downwards toward the floor
ii. the light emitted may have microbicidal and/or germicidal and/or viracidal and/or antimicrobial properties and
iii. the light emitted may be within, but not limited exclusively to, the ultraviolet spectrum
iv. the light emitted may also be of any suitable intensity and power to effect microbicidal and/or germicidal and/or viracidal and/or antimicrobial properties
v. the pressure triggered switches and shining LED lights/light source are confined to the silhouette of the shoe footprint to provide concealment of light that is dangerous to the human or animal eye and skin
vi. when weight is taken off the pressure sensor while walking or lifting the foot, the pressure switch is turned off, breaking the electronic circuit and ceasing current flow through the light source, thereby turning off the LEDs/light source
vii. alternatively the switch for the LEDs/light source may be triggered by any other method such as galvanic skin sensing, light occlusion, moisture detection, proximity detection, infrared sensing or any other suitable means.
b. Alternatively an individual using the invention in the form of a floor mat may simultaneously sterilize the opposing surfaces of the floor and sole of the shoe which are in direct contact with one another, such that:
i. the weight of the operator triggers a collection of small switches within the flooring mat/tile, which
ii. completes an electronic circuit to provide current to
iii. Light Emitting Diodes (LEDs) or a light source embedded within the floor mat and shine directly upward
iv. the light emitted has microbicidal and/or germicidal and/or viracidal and/or antimicrobial properties and
v. the light emitted may be within, but not limited exclusively to, the ultraviolet spectrum,
vi. the light emitted may also be of any suitable intensity and power to effect microbicidal and/or germicidal and/or viracidal and/or antimicrobial properties
vii. because each LED/light source unit has an independent pressure sensor switch in this embodiment, only LEDs/light source units in direct contact with a shoe will illuminate, thus allowing tailored confinement of the sterilizing light specifically to the silhouette of the shoe sole standing above it
viii. when weight is taken off the pressure sensor while walking or lifting the foot, the pressure switch is turned off, breaking the electronic circuit and ceasing current flow through the light source, thereby turning off the LEDs/light source
ix. alternatively the switch for the LEDs/light source may be triggered by any other method such as galvanic skin sensing, light occlusion, moisture detection, proximity detection, infrared sensing or any other suitable means.

2. A device composed of:
a. A sanitizing surface automatically sensitive to pressure and touch which is placed on the sole of a shoe and further composed of:
i. an illuminating surface when activated shines down toward the ground
ii. the same illuminating surface further composed of multiple LEDs/light sources emitting light in the UV wavelength or any wavelength capable of inducing DNA and/or RNA and/or protein damage and/or microbicidal, viracidal and/or antibacterial and/or antimicrobial properties
iii. an electronic circuit connected to LEDs/light sources which may be placed in a series or parallel fashion
iv. a power source which may be portable and in the form of a battery
v. a switch sensitive to weight
vi. a switch connected to the said circuit which when deployed lights the LEDs and illuminating surface
vii. alternatively any suitable light source emitting ultraviolet and/or or microbicidal, viracidal, antibacterial, antimicrobial properties may be used
viii. alternatively a switch for the LEDs/light source which may be triggered by any other method such as galvanic skin sensing, light occlusion, moisture detection, proximity detection, infrared sensing or any other suitable means to detect the operator's fully planted weight on the ground.
b. Alternatively the device may be placed on the floor in the shape of a mat or tile such that:
i. the mat or tile is embedded with a matrix of individual light sources/LEDs, which when stepped upon deploys a switch to emit light, but only in those regions in which the shoe is in direct contact with
ii. the illuminating surface when deployed shines upward toward the sole of the shoe standing upon it
iii. all LED/light source units, however, may be connected to the same power source but in the form of a parallel array-like fashion
iv. alternatively a switch for the LEDs/light source which may be triggered by any other method such as galvanic skin sensing, light occlusion, moisture detection, proximity detection, infrared sensing or any other suitable means to detect the operator's fully planted weight on the ground
v. alternatively the device in the form of a floor mat/tile may be modified to detect the present of liquid or dropped contamination material on the surface of the floor mat/tile.
c. Alternatively the invention may be practiced in both the form of a shoe and floor mat/tile simultaneously.

3. The device, method and material composition may be:
a. modified by any suitable means to fulfill the same intended function
b. modified with any suitable material to fulfill the same intended function
c. modified and ruggedized and/or modified for stealth for military defense and biological warfare applications
d. utilized for human or animal use
e. utilized on surfaces other than the floor
f. utilized in fields other than medicine, requiring sterile and/or infection, contamination control.
